(19) United States
(12) Patent Application Publication
(10) Pub. No.: US 2013/0225971 A1
(43) Pub. Date: Aug. 29, 2013

(54) PHOTOTHERAPY APPARATUS

(71) Applicant: Shuhei Tanaka, Kanagawa (JP)

(72) Inventor: Shuhei Tanaka, Kanagawa (JP)

(73) Assignee: PANASONIC CORPORATION, Osaka (JP)

(21) Appl. No.: 13/883,117

(22) PCT Filed: Oct. 27, 2011
(86) PCT No.: PCT/JP2011/006017

§ 371 Date: May 2, 2013

(30) Foreign Application Priority Data
Nov. 5, 2010 (JP) 2010-248077

(51) Int. Cl. A61B 5/00

(52) U.S. Cl. CPC A61B 5/4848
USPC 600/407

(57) ABSTRACT

The invention provides a phototherapy apparatus that allows confirmation that the therapeutic light has been irradiated on the appropriate site for treatment and as a result, is able to perform appropriate treatment. This phototherapy apparatus is provided with a photoirradiation part configured of a transparent material with a first surface whereon the site to be treated is placed and a second surface that is back to back with the first surface; a light source that outputs the therapeutic light and shines the therapeutic light into the photoirradiation part; and a photographing part with sensitivity in the wavelength region of the therapeutic light. The photographing part is configured to photograph the second surface.

### TECHNICAL FIELD

The present Invention relates to a phototherapy apparatus.

### BACKGROUND ART

A phototherapy apparatus of the related art has a configuration in which therapeutic light from a light source is irradiated onto an affected part directly or through a light guide in a noncontact state (see PTL 1). In the phototherapy apparatus, an intended disease differs depending on the wavelength of therapeutic light. For example, when infrared light (about 700 nm to 2500 nm) used as therapeutic light, this is used for relief of pain due to chronic non-infectious inflammation of a muscle or joint or for treatment of a joint with rheumatism developed. When ultraviolet light (about 280 nm to 400 nm) is used as therapeutic light, this is used for treatment of refractory skin disease, such as psoriasis vulgaris, atopic dermatitis, vitiligo vulgaris (vitiligo), alopecia areata, prurigo, or pustulosis palmoplantaris, or athletes foot (see PTL 2). When blue light (about 400 nm to 500 nm) is used as therapeutic light, this is used for treatment of skin disease, such as acne or seborrhea developed in a face, a chest, or a back (for example, see PTL 3),

In this way, while the phototherapy apparatus is useful for treatment of various diseases, treatment by the phototherapy apparatus may not be preferable depending on a site onto which light is irradiated. For example, infrared light having significant power is used for relief of pain due to chronic non-infectious inflammation of a muscle or joint or for treatment of a joint with rheumatism developed. It is not preferable that infrared light having significant power is irradiated onto the eye. Ultraviolet light which is used for treatment of refractory skin disease or athlete's foot causes cataract when being irradiated onto the eye. Ultraviolet light causes skin cancer when being irradiated onto the skin. Blue light which is used for treatment of skin disease, such as acne or seborrhea, causes blue light retina damage as serious disorder of the eye when being irradiated onto the eyes or physiological damage to the skin.

Accordingly, a phototherapy apparatus which suppresses irradiation of therapeutic light onto the eye or an unwanted part is suggested (for example, see PTL 4), in the phototherapy apparatus suggested in PTL 4, visible light is input inside a photoirradiation section constituted by a light guide member, and an affected part is brought into contact with the surface of the photoirradiation section to perform phototherapy. That is, in a state where a living body is not in contact with the surface of the photoirradiation section, the emission of light from the inside of the photoirradiation section is suppressed; and when a living body is in contact with the surface of the photoirradiation section, light is emitted from the contact portion toward the living body.

With this configuration, it is possible to suppress the situation in which unwanted light which does not contribute to treatment leaks from the photoirradiation section and is carelessly irradiated onto the eye of a patient. Since light is irradiated only onto a site to be treated in contact with the surface of the photoirradiation section, it is also possible to suppress irradiation of light onto an unwanted part.

### CITATION LIST

### Patent Literature

PTL 1
Japanese Patent Application Laid-Open No. SHO 63-21069
PTL 2
Japanese Patent Application Laid-Open No. 2007-159785
PTL 3
Japanese Translation of PCT Application Laid-Open No. 2002-526128
PTL 4
Japanese Patent Application Laid-Open No. 2009-95549

### SUMMARY OF INVENTION

### Technical Problem

If a light source of the phototherapy apparatus suggested in PTL 4 is a light source of infrared light or ultraviolet light, for example, relief of pain due to chronic non-infectious inflammation of a muscle or joint, or treatment of a joint with rheumatism developed, or disease, such as refractory skin disease or athletes foot becomes possible. On the other hand, in the ease of infrared light or ultra-violet light, it is not possible to confirm that therapeutic light is irradiated onto an appropriate site to be treated. For this reason, it may he difficult to perform appropriate treatment.

That is, since infrared light or ultraviolet light is light in an invisible wavelength band, it is not possible to confirm whether or not light is irradiated onto an appropriate site to be treated. In this way, since it is not possible to confirm a site onto which therapeutic light is irradiated and the photoirradiation state, it may be difficult to understand the treatment effect, and appropriate treatment may not be performed. In particular, since ultraviolet light causes damage to the skin, it is necessary to suppress irradiation onto an unwanted part ac much as possible. For this reason, it is very important to understand a site onto which light is irradiated and the photoirradiation state.

If a light source of the phototherapy apparatus suggested in PTL 4 is a light source of blue light, for example, treatment of skin disease, such as acne or seborrhea, becomes possible. Since blue light is visible light, it is possible to visually recognize that therapeutic light is irradiated onto a site to be treated. However, as described above, if the influence of blue light on a living body, such as blue light retina damage or physiological damage to the skin, is taken into consideration, it is not preferable to make confirmation with the eyes directly.

Accordingly, an object of the invention is to provide a phototherapy apparatus which can safely confirm whether or not therapeutic light is irradiated onto an appropriate site for treatment, thereby performing appropriate treatment.

### Solution to Problem

In order to attain this object, a phototherapy apparatus of the invention includes 1) a photoirradiation section that has a first surface on which a site to be treated is disposed and a second surface opposite to the first surface, and is constituted by a light guide member, 2) a light source section that outputs therapeutic light and inputs therapeutic light inside the photoirradiation section, and 3) a photographing section that has sensitivity in the wavelength band of therapeutic light. The photographing section photographs the second surface. Therefore, a desired object is attained.

### Advantageous Effects of Invention

Since the phototherapy apparatus of the invention has the photographing section which has sensitivity in the wavelength band of therapeutic light, an image imaged by the photographing section is confirmed, whereby it is possible to confirm whether or not therapeutic light is safely irradiated onto an appropriate site to be treated. Furthermore, when the image imaged by the photographing section is displayed on the display section, it is possible to more simply understand a site onto which therapeutic light is irradiated and the photoirradiation state only by confirming the display section. As a result, it is possible to perform appropriate treatment.

Further, it may not be preferable that light, such as infrared light, ultraviolet light, or blue light, which is used for phototherapy is irradiated onto the eye directly or is irradiated onto an unwanted pail. In contrast, according to the phototherapy apparatus of the invention, it is possible to understand the photoirradiation state through the display section, and to realize phototherapy with consideration for safety of the user, the patient, or the like.

When therapeutic light is invisible light (infrared light or ultraviolet light), in the phototherapy apparatus of the related art, the patient has difficulty in realizing that he/she is receiving phototherapy. In contrast, in the phototherapy apparatus of the invention, it is possible to confirm the photoirradiation state by confirming the display section, whereby the patient can realize that he/she is receiving treatment.

### BRIEF DESCRIPTION OF DRAWINGS

FIG. 1 is a perspective view of a phototherapy apparatus according to Embodiment 1 of the invention.

FIG. 2 is a diagram showing the usage state of the phototherapy apparatus according to Embodiment 1 of the invention.

FIG. 3 is a front-side perspective view of the phototherapy apparatus according to Embodiment 1 of the invention.

FIG. 4 is a lateral-side perspective view of the phototherapy apparatus according to Embodiment 1 of the invention.

FIG. 5 is a sectional side view showing the usage state of the phototherapy apparatus according to Embodiment 1 of the invention.

FIG. 6 is a sectional side view of a finger photoirradiation section of the phototherapy apparatus according to Embodiment 1 of the invention.

FIG. 7 is a top view of a finger photoirradiation section of the phototherapy apparatus according to Embodiment 1 of the invention.

FIG. 8 is a block diagram of the phototherapy apparatus according to Embodiment 1 of the invention.

FIG. 9 is an operation flowchart of the phototherapy apparatus according to Embodiment 1 of the invention.

FIGS. 10A to 10C is a diagram showing a finger image displayed on a display section of the phototherapy apparatus according to Embodiment 1 of the invention.

FIGS. 11A and 11B are diagrams showing the disposition of cameras provided on a substrate surface of a phototherapy apparatus according to Embodiment 2 of the invention.

### DESCRIPTION OF EMBODIMENTS

A phototherapy apparatus of the invention includes 1) a photoirradiation section, 2) a light source section, and 3) a photographing section, and preferably, further includes 4) a display section.

The photoirradiation section has a first surface on which a site to be treated is disposed such that the site to be treated is brought into contact with the first surface. A patient brings an affected part into contact with the first surface, and light which is guided by the photoirradiation section is irradiated onto the affected part in contact with the first surface. The photoirradiation section has a second surface which is opposite to the first surface. The second surface is photographed by the photographing section.

The shape of the first surface of the photoirradiation section is preferably set such that the affected part is easily brought into contact therewith. Specifically, the first surface of the photoirradiation section is preferably a curved surface, and may be a hemispherical surface, a semi-cylindrical surface, or the like. The shape of the photoirradiation section is not particularly limited, and may be a hollow hemispherical shape or a hollow semi-cylindrical shape. The outer surface having a hollow hemispherical shape or a semi-cylindrical shape may he the first surface, and the inner surface having a hollow hemispherical shape or a hollow semi-cylindrical shape may be the second surface.

The photoirradiation section is a light guide member which can guide therapeutic light, and is preferably a member which is transparent for therapeutic light. Examples of a transparent material for the light guide member of the photoirradiation section include transparent acrylic resin (refractive index of 1.49), transparent glass (refractive index of 1.51), purfluororesin (refractive index of 1.34), and the like.

The photoirradiation section has an entrance through which light output from the light source section is input to the light guide member. Light input through the entrance is guided by the light guide member of the photoirradiation section; and is emitted from a portion in contact with the affected part in the first surface of the photoirradiation section. Emitted light is irradiated onto the affected part.

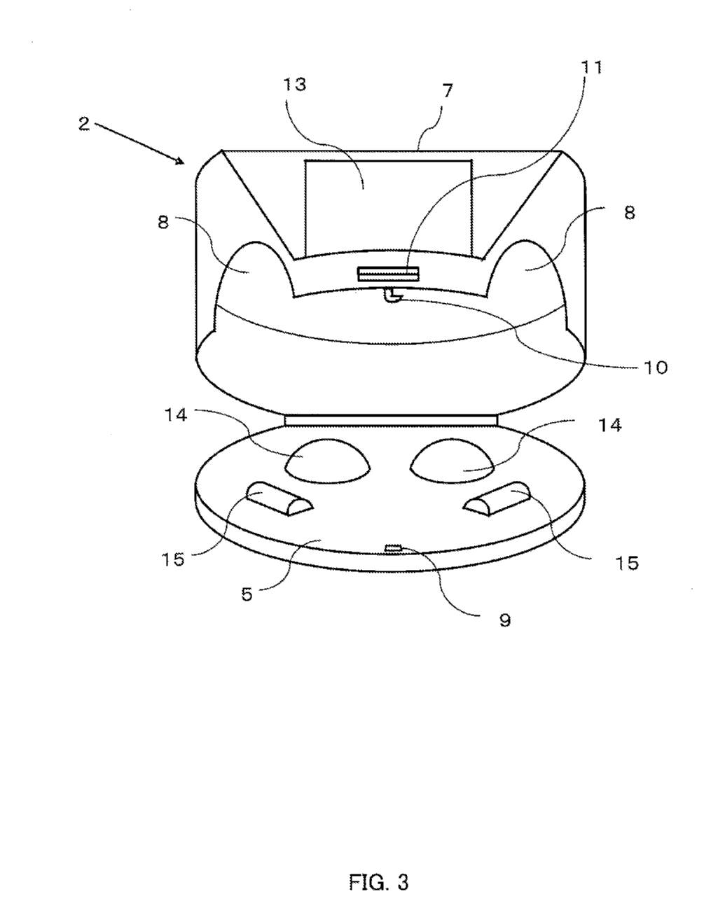

A single phototherapy apparatus may include one photoirradiation section or may include a plurality of photoirradiation sections. For example, a phototherapy apparatus shown in FIG. 3 includes two finger photoirradiation sections 14 and two wrist photoirradiation sections 15.

The light source section is a member which outputs light (therapeutic light) used for treatment. Therapeutic light differs depending on an object to be treated by the phototherapy apparatus, and is preferably at least one of infrared light, ultraviolet light, and blue light. The light source section may output light of two or more wavelengths. Infrared light (about 700 nm to 2500 nm) is used for relief of pain or for treatment of a joint with rheumatism developed; ultraviolet light (about 280 nm to 400 nm) is used for treatment of refractory skin disease, such as psoriasis vulgaris, atopic dermatitis, vitiligo vulgaris (vitiligo), alopecia areata, prurigo, or pustulosis palmoplantaris, or athlete's foot; and blue light (about 400 nm to 500 nm) is used for treatment of skin disease, such as acne or seborrhea developed in a face, a chest, or a back.

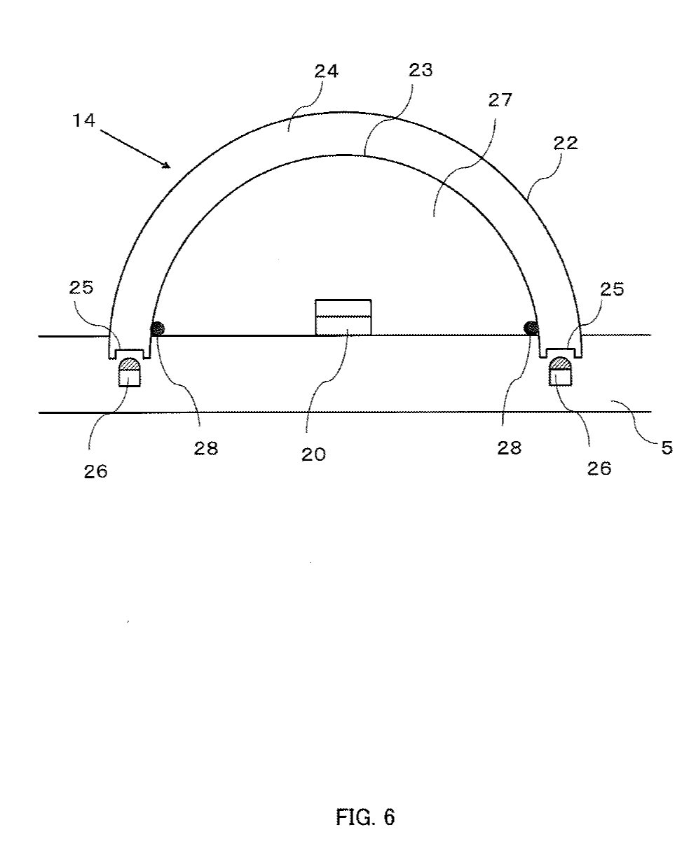

It is preferable that the light source section is disposed such that light output from the light source section is input to the light guide member of the photoirradiation section through the entrance of the photoirradiation section. The position that the light source section is disposed is not particularly limited, and when the photoirradiation section has a hollow hemispherical shape, entrances 25 may be provided in the notched end portions of the hollow hemisphere and light source sections 26 may he disposed to face the entrances (see FIG. 6).

The phototherapy apparatus has one or a plurality of light source sections for one photoirradiation section. Specifically, the phototherapy apparatus has light source sections corresponding to the number of entrances of the photoirradiation section.

The photographing section has sensitivity in the wavelength band of therapeutic light output from the light source section. The photographing section can image the second surface. The second surface is imaged during treatment, whereby it is possible to confirm a state of photoirradiation onto the site to be treated. Light which is guided by the light guide member of the photoirradiation section is irradiated only onto the affected part (site to be treated) in contact with the first surface, and other kinds of light are guided by the light guide member. For this reason, if the photographing section images the second surface, an image in which only the affected part with light irradiated is projected bright is obtained. Therefore, it is possible to confirm whether or not light is irradiated onto the affected part by observing the image imaged by the photographing section.

The position that the photographing section is disposed is not particularly limited insofar as the second surface of the photoirradiation section can be imaged, and the image section may be usually disposed to face the second surface. For example, when the shape of the photoirradiation section is a hollow hemispherical shape or a hollow semi-cylindrical shape, it is preferable that the photographing section is disposed inside the hollow portion of the photoirradiation section. Specifically, the photoirradiation section having a hollow hemispherical shape or a hollow semi-cylindrical shape is disposed on a substrate, and a space is defined between the substrate surface and the inner surface of the hollow hemispherical shape or the hollow semi-cylindrical shape of the photoirradiation section. It is preferable that photographing section 20 is disposed inside the defined space (see FIG. 6).

At this time, it is preferable that the defined space is a sealed space. The penetration of moisture into the space is suppressed, thereby preventing dimming of the photographing section or the photoirradiation section.

The phototherapy apparatus of the invention may further have a display section. An image imaged by the photographing section can be displayed on the display section. A user of the phototherapy apparatus confirms the image displayed on the display section, thereby confirming the irradiation state of therapeutic light onto the affected part (a site in contact with the first surface of the photoirradiation section).

Hereinafter, an embodiment of the phototherapy apparatus of the invention will he described in detail with reference to the drawings.

### Embodiment 1

In Embodiment 1 the configuration of a phototherapy apparatus which treats a finger joint and a wrist joint with rheumatoid arthritis developed. An affected part defined herein is a site where phototherapy is required, and in Embodiment 1, refers to a finger joint and a wrist join. A site to be treated is a living body site including the affected part, and in Embodiment 1, a finger including a finger joint and a wrist including a wrist joint.

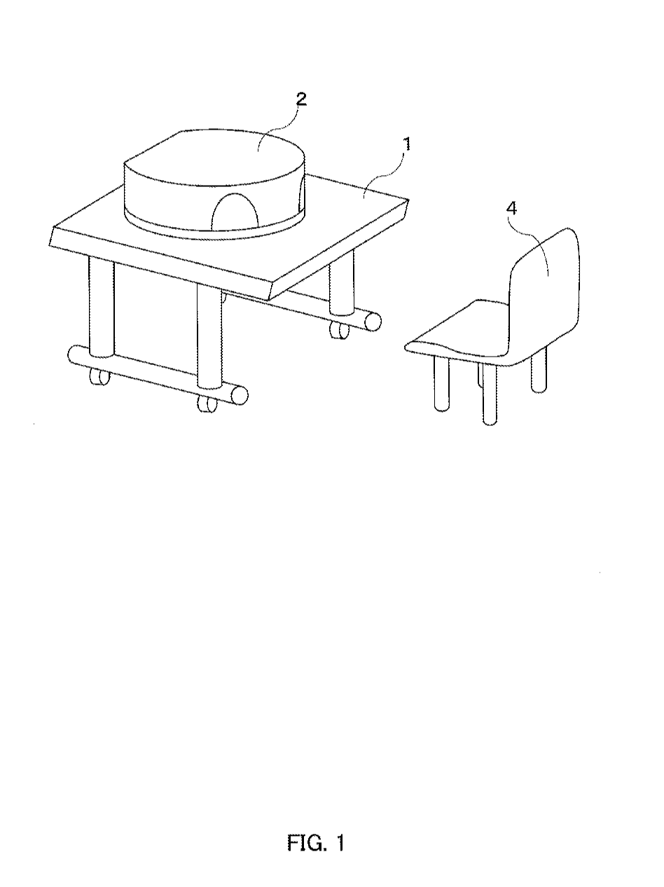

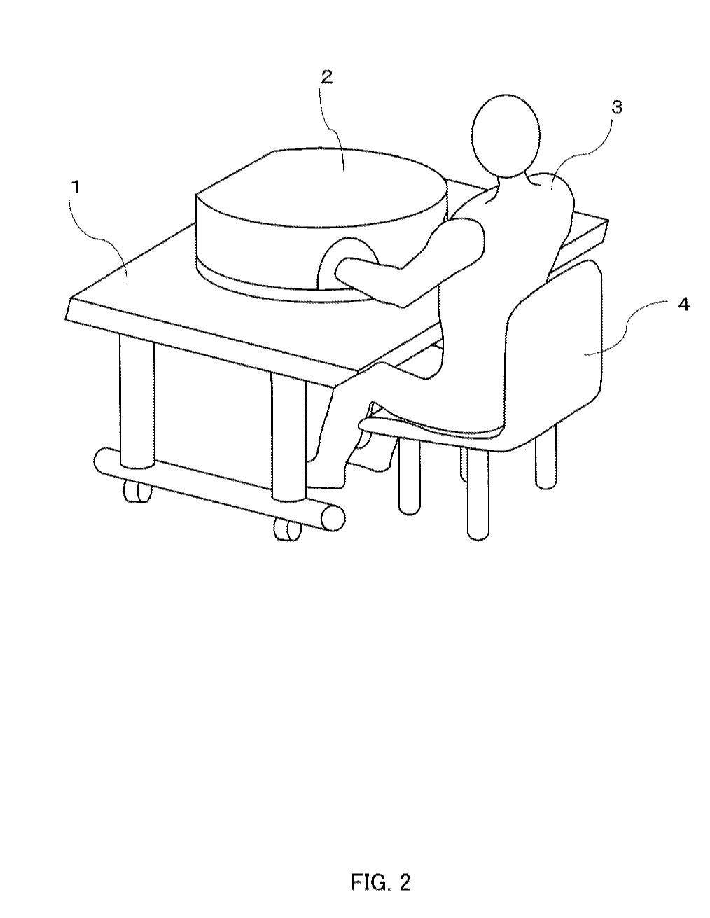

FIG. 1 is a perspective view of a phototherapy apparatus according to Embodiment 1 of the invention, and phototherapy apparatus s placed on desk 1. As shown in FIG. 2, patient 3 sits in chair 4, and phototherapy of the finger and wrist joints of the patient with rheumatism developed is performed using the phototherapy apparatus at home or hospital.

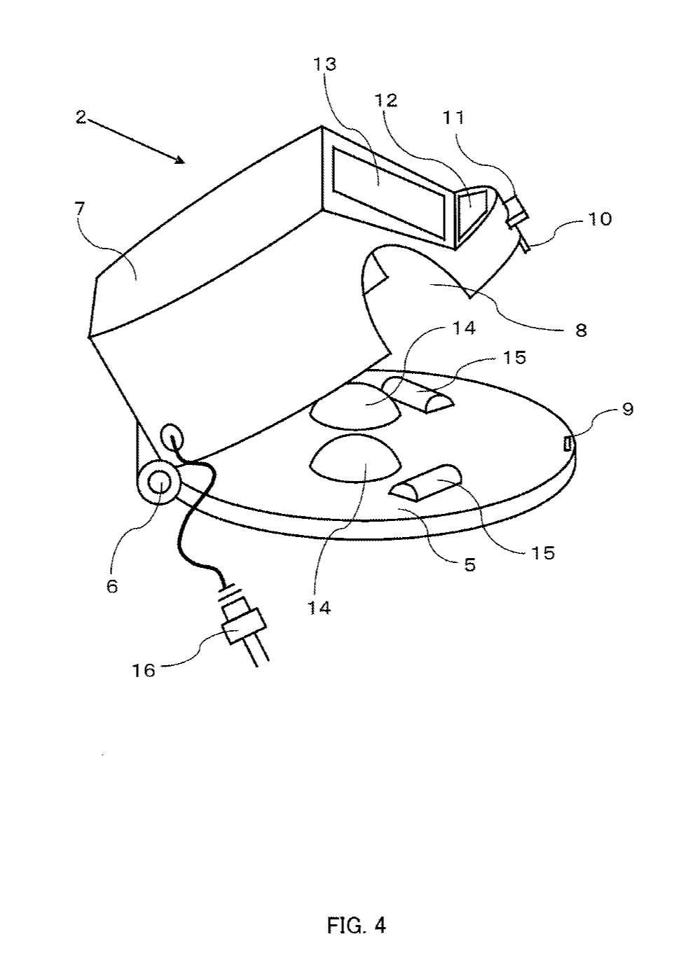

As shown in FIGS. 3 and 4, phototherapy apparatus 2 has disc-like substrate 5, and cover 7 which openably and closably covers the surface of substrate 5 by hinge 6. Cover 7 has a capped cylindrical shape with an open lower surface. Insertion slots 8 of the site to be treated are provided on the front side of the outer circumferential surface of cover 7, that is, on the left and right of the side opposite to hinge 6. Hook 10 which is engaged with engagement hole 9 of substrate 5 to close cover 7 is provided between left and right insertion slots 8 on the front side of the outer circumferential surface of cover 7. Hook button 11 which opens cover 7 is provided above hook 10.

Operating section 12 which is used to operate phototherapy apparatus 2 and display section 13 which displays the inside of phototherapy apparatus 2 are provided on the surface of cover 7. Two left and right finger photoirradiation sections 14 and two left and right wrist photoirradiation sections 15 are disposed at a predetermined interval on the surface of substrate 5. Specifically, wrist photoirradiation sections 15 are disposed between insertion slots 8 and finger photoirradiation sections 14 on the surface of substrate 5. Power is supplied to phototherapy apparatus 2 through power cord 16.

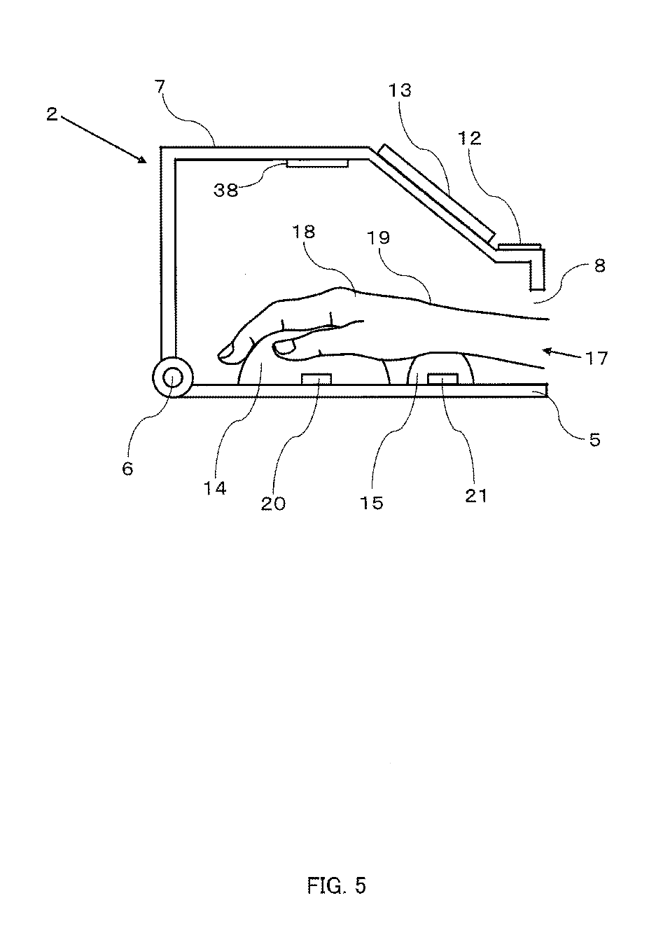

FIG. 5 is a sectional view of phototherapy apparatus 2 showing the usage state of phototherapy apparatus 2 of Embodiment 1. When using phototherapy apparatus 2, hand 17 which is a site to be treated is inserted from insertion slot 8, fingers 18 are placed on the surface of finger photoirradiation section 14, and wrist 19 is placed on the surface of wrist photoirradiation section 15. That is, in Embodiment 1, the outer surface of finger photoirradiation section 14 becomes a first surface on which fingers 18 are placed; and the outer surface of finger photoirradiation section 15 becomes a first surface on which wrist 19 is placed.

The outer surface (first surface) of finger photoirradiation section 14 substantially is a hemispherical surface, and the inside of finger photoirradiation section 14 is hollow. That is, finger photoirradiation section 14 has a hollow hemispherical shape. The outer surface (first surface) is substantially a hemispherical surface, whereby a finger which is a site to he treated is easily placed.

The outer surface of wrist photoirradiation section 15 has a linear shape in a direction (hereinafter, referred to as a left-right direction) perpendicular to a line from insertion slot 8 toward finger photoirradiation section 14; and a cross-section in a direction (hereinafter, referred to as a front-back direction) from insertion slot 8 toward finger photoirradiation section 14 has a semicircular shape. That is, the outer surface of wrist photoirradiation section 15 is a semi-cylindrical surface (semi-cylindrical outer surface). The inside of wrist photoirradiation section 15 is also hollow, and wrist photoirradiation section 15 has a hollow semi-cylindrical shape.

Finger photoirradiation section 14 and wrist photoirradiation section 15 have a role as a photoirradiation section which irradiates infrared light and a role of placing a site to be treated. Infrared light as therapeutic light is irradiated onto a site to be treated which is disposed on the outer surface of the photoirradiation section.

Finger photographing camera 20 and wrist photographing camera 21 are provided inside the hollow portions of finger photoirradiation section 14 and wrist photoirradiation section 15 so as to photograph the irradiation state of therapeutic light onto fingers 18 and wrist 19 in contact with finger photoirradiation section 14 and wrist photoirradiation section 15.

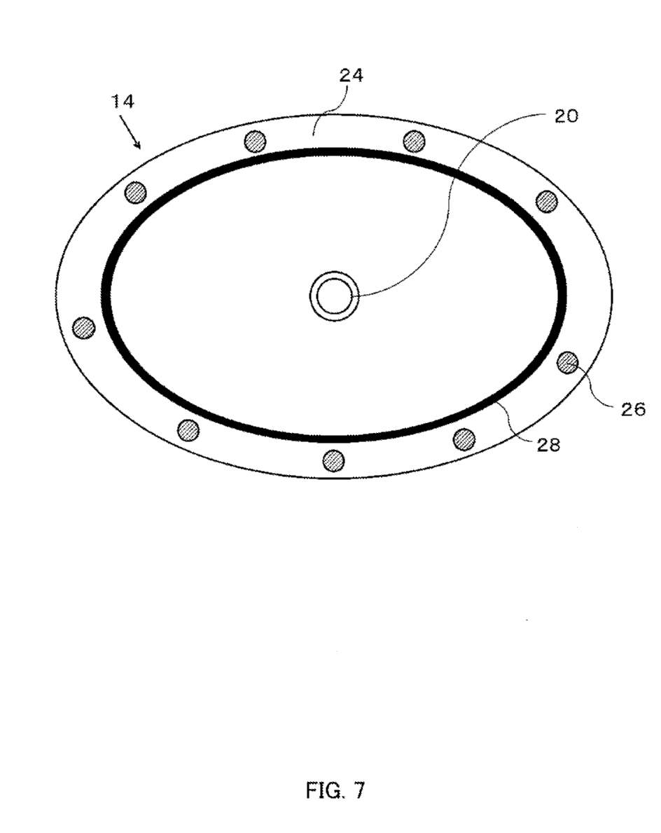

FIGS. 6 and 7 are a sectional view and a top view of finger photoirradiation section 14. As described above, since finger photoirradiation section 14 has a hollow hemispherical shape, the outer surface of finger photoirradiation section 14, that is, the surface (hereinafter, referred to as placement surface 22) on which fingers 18 as a site to be treated are placed is substantially a hemispherical surface. The inner surface of finger photoirradiation section 14 is the surface (hereinafter, referred to as opposite surface 23) which is opposite to placement surface 22. Similarly to placement surface 22, opposite surface 23 is substantially a hemispherical surface.

Finger photoirradiation section 14 is formed of a light guide member, and finger photoirradiation section 14 itself has light guiding property for light (therapeutic light) output from light source 26. That is, a portion between placement surface 22 and opposite surface 23 has a role as light guide 24 which transmits light. As the light guide member, for example, transparent acrylic resin (refractive index of 1.49), transparent glass (1.51), perfluororesin (refractive index of 1.34), or the like may be used.

The thickness (the distance between placement surface 22 and opposite surface 23) of light guide 24 constituting finger photoirradiation section 14 is substantially uniform. A plurality of entrances 25 through which light is input to light guide 24 are provided in the end surfaces of light guide 24 as the lower opening end of finger photoirradiation section 14 having a hollow hemispherical shape. As shown in FIG. 6, the lower end portions of finger photoirradiation section 14, that is, the end portions including entrances 25 disposed inside substrate 5 are embedded in substrate 5.

A plurality of light sources 26 corresponding to the number of entrances 25 are disposed (embedded) in substrate 5. Each light source 26 is disposed such that irradiated light is input to light: guide 24 through entrance 25. Light source 26 irradiates therapeutic light. It is preferable that therapeutic light for phototherapy of finger and wrist joints of a patient with rheumatism developed is near infrared light having a wavelength band of about 700 nm to 2000 nm.

Light which is output from light source 26 and input from entrance 25 of finger photoirradiation section 14 to light guide 24 can be intensively irradiated onto a portion (site to be treated) which is in contact with the outer surface of finger photoirradiation section 14. That is, while light from the inside of finger photoirradiation section 14 is irradiated onto a living body site (site to be treated) in contact with placement surface 22; on the other hand, photoirradiation onto a living body site not in contact with placement surface 22 is significantly suppressed. Therefore, a finger joint as an affected part is brought into contact with placement surface 22, whereby therapeutic light can be selectively irradiated onto the affected part.

The reason that therapeutic light can be selectively irradiated onto the affected part in contact with placement surface 22 will be described. For example, it is assumed that the light guide member of finger photoirradiation section 14 is made of acrylic resin. When a site to be treated including an affected part is not in contact with placement surface 22 of finger photoirradiation section 14, therapeutic light which is guided by the light guide member of finger photoirradiation section 14 is input from acrylic resin (refractive index of 1.49) to the air (refractive index of 1.00). At this time, the critical angle of the interface of light emitted from the light guide member of finger photoirradiation section 14 becomes 42.155 degrees. For this reason, if therapeutic light which is guided by light guide 24 reaches placement surface 22 or opposite surface 23 inside finger photoirradiation section 14 at an incident angle equal to or greater than 42.155 degrees, the light is totally reflected by placement surface 22 or opposite surface 23 inside finger photoirradiation section 14. Then, therapeutic light is not emitted outside finger photoirradiation section 14, and total reflection is repeated inside light guide 24 of finger photoirradiation section 14.

Meanwhile, when a site to be treated including an affected part is in contact with placement surface 22 of finger photoirradiation section 14, the reflection state of therapeutic light in the contact portion with placement surface 22 differs. That is, when a site to be treated is in contact, therapeutic light which is guided by the light guide member of finger photoirradiation section 14 is input from acrylic resin (refractive index of 1.49) to a skin of a living body (refractive index of 1.4). A this time, the critical angle of the interface of light emitted from the light guide member of finger photoirradiation section 14 becomes 69.984 degrees.

In this way, a site to be treated including an affected part is brought into contact with placement surface 22, whereby the critical angle of the interface of therapeutic light emitted from the light guide member of finger photoirradiation section 14 increases (changes from 42.155 degrees to 69.984 degrees), and therapeutic light easily leaks from a portion which is brought into contact with the site to be treated. Accordingly, therapeutic light can be selectively emitted from placement surface 22 with which the site to be treated is in contact, and therapeutic light can be intensively irradiated onto the affected part. Therefore, phototherapy with high energy efficiency can he carried out.

In addition, it is possible to suppress the irradiation of therapeutic light leaking outside phototherapy apparatus 2 onto the eye of a patient or an operator, thereby safely carrying out treatment.

Next, as a principal feature of the invention, a configuration for confirming the state of photoirradiation onto the site to be treated placed in the photoirradiation section will be described.

Finger photographing camera 20 is disposed inside the space (hereinafter, referred to as hollow portion 27) partitioned by the surface of substrate 5 and opposite surface 23 on the surface of substrate 5. Finger photographing camera 20 is disposed in a direction such that opposite surface 23 can be photographed.

Finger photographing camera 20 is provided for the purpose of observing the site to he treated onto which therapeutic light from light source 26 is irradiated. That is, finger photographing camera 20 photographs the site to he treated disposed on placement surface 22 through opposite surface 23. Accordingly, it is necessary to form the light guide member of a substantially transparent material. Since therapeutic light from light source 26 is infrared light, finger photographing camera 20 should have sensitivity in at least an infrared light band.

It is preferable that hollow portion 27 partitioned by the surface of substrate 5 and opposite surface 23 of finger photoirradiation section 14 is substantially in a sealed state. In order to place hollow portion 27 in the sealed state, packing 28 is provided in the portion in which finger photoirradiation section 14 is provided on the surface of substrate 5 (see FIG. 6). Since light source 26 of phototherapy apparatus 2 should output intense light for a comparatively long time, at the time of driving of phototherapy apparatus 2, heat is generated from the apparatus itself. If the temperature of hollow portion 27 is raised with generated heat, moisture easily enters hollow portion 27. Moisture which enters hollow portion 27 may cause dimness on opposite surface 23 of finger photoirradiation section 14 or finger photographing camera 20. The occurrence of dimness may affect the photographed image of finger photographing camera 20. For this reason, it is preferable that hollow portion 27 is placed in the sealed state, thereby suppressing the penetration of moisture.

Dimness on opposite surface 23 means that water droplets arc stuck to the surface. If water droplets are stuck to the surface of opposite surface 23, therapeutic light easily leaks from the light guide member to the outside. That is, since the critical angle of the interface of light input from the light guide member of finger photoirradiation section 14 to water is greater than the critical angle of the interface of light input from the light guide member of finger photoirradiation section 14 to the air, therapeutic light easily leaks from opposite surface 23, on which dimness occurs, to the outside. For this reason, it is preferable that packing 28 or the like is provided, thereby substantially placing hollow portion 27 in the sealed state.

in FIGS. 6 and 7, the configuration of finger photoirradiation section 14 is shown. Wrist photoirradiation section 15 is different in shape from linger photoirradiation section 14, however, a basic configuration is identical. That is, therapeutic light is selectively irradiated onto the site to be treated in contact with the outer surface of wrist photoirradiation section 15, and whether or not therapeutic light is irradiated onto the affected part is confirmed by the image photographed by the photographing camera. Similarly to finger photoirradiation section 14, a photographing camera is disposed inside the hollow portion of wrist photoirradiation section 15, and the inside of the hollow portion is substantially placed in a sealed state. Accordingly, detailed description of wrist photoirradiation section 15 will not be repeated.

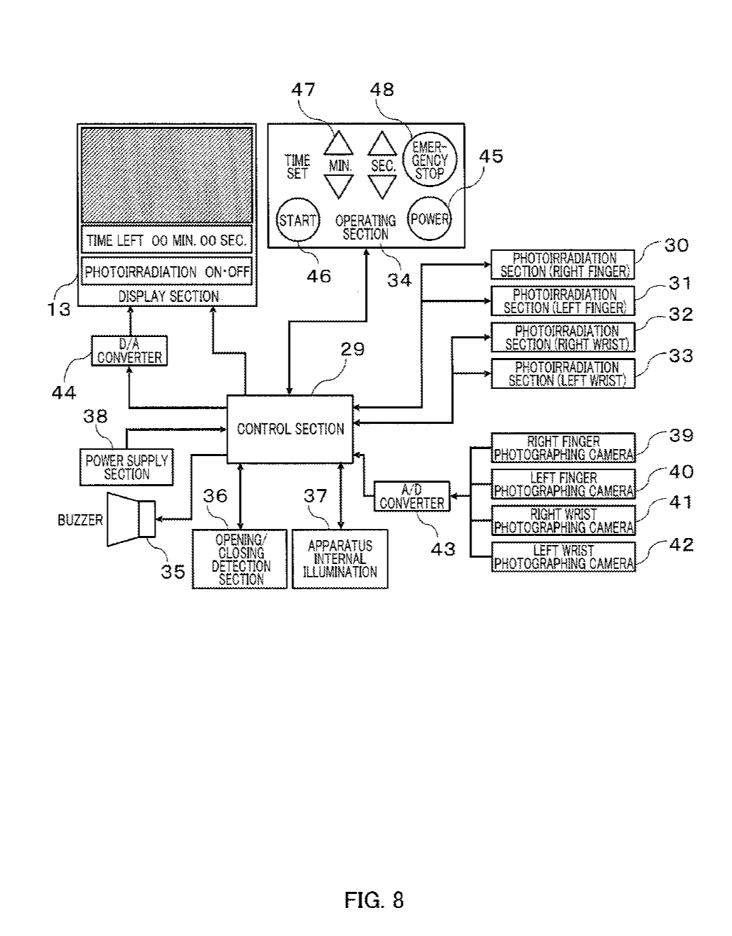

FIG. 8 is a block diagram of phototherapy apparatus 2 according to Embodiment 1 of the invention. Right finger photoirradiation section 30, left finger photoirradiation section 31, right wrist photoirradiation section 32, left wrist photoirradiation section 33, operating section 34, buzzer 35, opening/closing detection section 36, and apparatus internal illumination 37 are connected to control section 29. Display section 13 is connected to control section 29 through D/A converter 44. Power is supplied from power supply section 38 to each of these sections.

Right finger photographing camera 39, left finger photographing camera 40, right wrist photographing camera 41, and left wrist photographing camera 42 respectively provided in right finger photoirradiation section 30, left finger photoirradiation section 31, right wrist photoirradiation section 32, and left wrist photoirradiation section 33 are connected to control section 29 through A/D converter 43.

Control section 29 performs power and signal supply to a plurality of light sources 26 respectively provided in the photoirradiation sections (30 to 33). Operating section 34 is provided on the front side (insertion slot 8 side) of the upper surface of cover 7 (see FIG. 4), and includes power switch 45, start switch 46, treatment time input switch 47, and emergency stop switch 48. Display section 13 is provided in the upper surface of cover 7 (see FIG. 4), and can indicate whether or not photoirradiation is performed, indicate a remaining photoirradiation time, and display video of the photographing camera (39, 40, 41, and 42) provided in the photoirradiation section (30, 31, 32, and 33). The photographed image of the photographing camera is observed, whereby the photoirradiation state onto the affected part (left and right fingers and left and right wrists) can be confirmed.

Buzzer 35 emits warning sound or the like. Opening/closing detection section 36 detects a state where hook 10 is engaged with engagement hole 9 of substrate 5, and cover 7 is closed.

Apparatus internal illumination 37 is provided in a ceiling portion of the lower surface of cover 7 (see FIG. 5). Apparatus internal illumination 37 is turned on when therapeutic light is not irradiated. For this reason, when cover 7 is closed, the inside of phototherapy apparatus 2 or the position of the hand of the affected part is photographed by the photographing camera (39, 40, 41, and 42), and video is displayed on display section 13, whereby it is possible to confirm the inside of phototherapy apparatus 2 or the position of the hand of the affected part. It should suffice that apparatus internal illumination 37 is a general illumination which does not output therapeutic light (infrared light) but irradiates visible light, unlike light source 26.

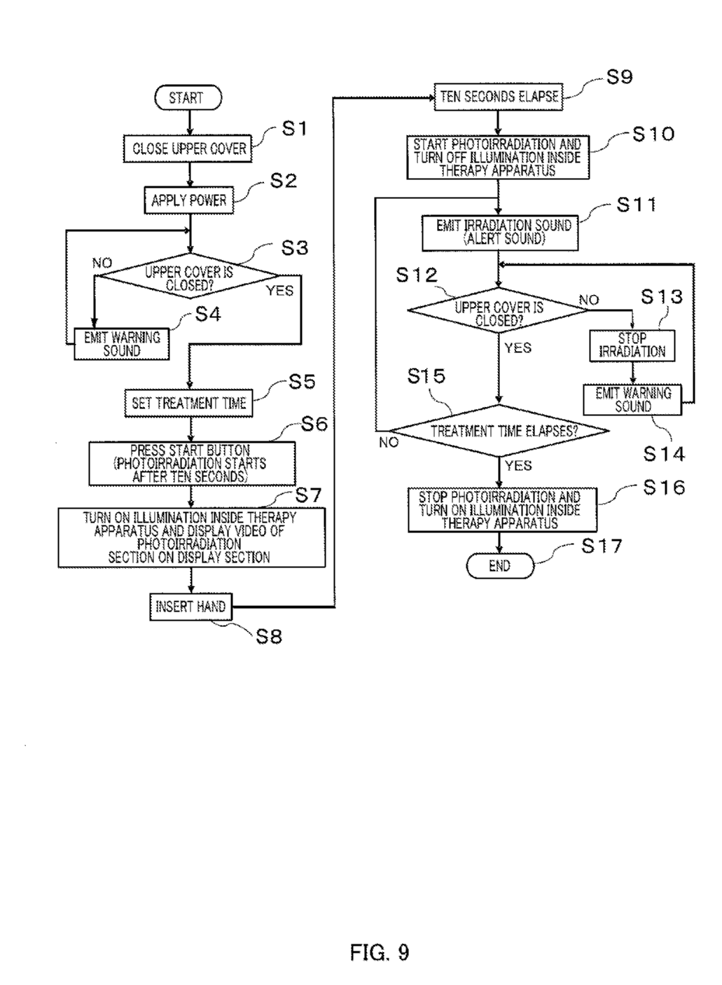

FIG. 9 is a flowchart showing a procedure of phototherapy by phototherapy apparatus 2 in the above configuration.

First, as shown in FIG. 1, substrate 5 is covered by cover 7 of FIGS. 3 and 4 (S1 of FIG. 9). Next, power switch 45 is pressed to apply power (52 of FIG. 9). When this happens, whether or not hook 10 is engaged with engagement hole 9 of substrate 5, and cover 7 is placed in the closed state is detected by opening/closing detection section 36 (S3 of FIG. 9). At this time, if cover 7 is not closed, a warning is emitted from buzzer 35 (S4 of FIG. 9). If cover 7 is closed, then, the treatment time is set by treatment time input switch 47 of operating section 34 (S5 of FIG. 9).

Thereafter, if start switch 46 of operating section 34 is pressed (S6 of FIG. 9), the respective photoirradiation sections (30, 31, 32, and 33) are driven after a predetermined standby time. After that, output of therapeutic light from light source 26 provided in the respective photoirradiation sections (30, 31, 32, and 33) toward light guide 24 becomes ready. If start switch 46 of operating section 34 is pressed, apparatus internal illumination 37 is turned on, and video from the photographing cameras (39, 40, 41, and 42) provided in the respective photoirradiation sections (30, 31, 32, and 33) is displayed on display section 13 (S7 of FIG. 9).

In the meantime, as shown in FIG. 2, patient 3 sits in chair 4, inserts the arms from insertion slots 8, and places the hands and wrists on the respective photoirradiation sections (30, 31, 32, and 33) (S8 of FIG. 9). Thereafter, if a predetermined standby time has elapsed (S9 of FIG. 9), the respective photoirradiation sections (30, 31, 32, and 33) are driven, therapeutic light is output from light sources 26, and apparatus internal illumination 37 is turned off (S10 of FIG. 9).

While light is irradiated from light sources 26 of the respective photoirradiation sections (30, 31, 32, and 33), buzzer 35 emits an alert sound, for example, pip, pip, at a given interval and notifies patient 3 that phototherapy is being performed to call for attention (S11 of FIG. 9). While light is irradiated from light sources 26, whether or not the cover 7 is closed is detected by opening/closing detection section 36 (S12 of FIG. 9). If cover 7 is opened, photoirradiation from all light sources 26 is stopped (S13 of FIG. 9), and the condition that cover 7 is opened is notified from buzzer 35 (S14 of FIG. 9).

While the output of therapeutic light from light sources 26 is being performed, insofar as the condition that cover 7 is continuously closed is detected by opening/closing detection section 36, the treatment time set by treatment time input switch 47 is reduced (S15 of FIG. 9). If the treatment time has elapsed, photoirradiation from light sources 26 is stopped, apparatus internal illumination 37 is turned on (S16 of FIG. 9), and the process ends (S17 of FIG. 9).

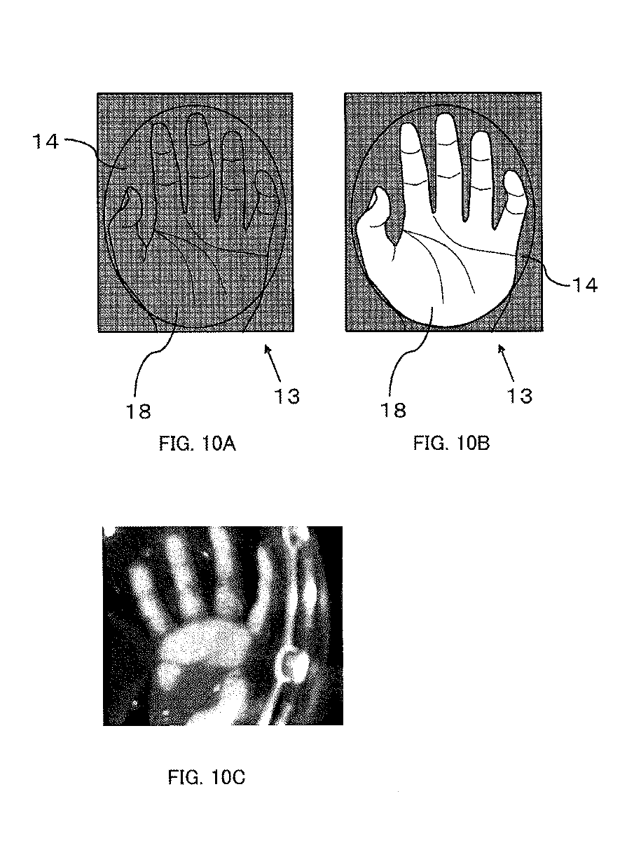

FIGS. 10A to 10C shows schematic views (FIGS. 10A and 10B) of the display section on which a finger image photographed by phototherapy apparatus 2 according to Embodiment 1 of the invention is displayed, and an actual photographed image (FIG. 10C) displayed on the display section.

FIG. 10A is a schematic view of an image of the finger photoirradiation section when infrared light is not irradiated from light source 26. As shown in FIG. 10A, dark image is displayed on the display section 13 as a whole.

In contrast, FIG. 10B is a schematic view of an image of the finger 1 0 photoirradiation section when infrared light is irradiated from light source 26. As shown in FIG. 10B, the site to be treated in contact with finger photoirradiation section 14 is displayed bright, and a portion not in contact with finger photoirradiation section 14 is displayed dark. In this way, it is confirmed that light is irradiated onto the site to be treated in contact with finger photoirradiation section 14. As described above, the site to be treated in contact with placement surface 22 of finger photoirradiation section 14 is displayed bright because therapeutic light is irradiated, and a portion not in contact with placement surface 22 is displayed dark because therapeutic light is poorly irradiated.

FIG. 10C shows an image which is actually photographed by irradiating infrared light from light source 26. As shown in FIG. 10C, a situation in which therapeutic light is irradiated onto the site to be treated in contact with placement surface 22 of finger photoirradiation section 14 is clearly shown. That is, it is understood that a portion in closer contact with placement surface 22 is displayed brighter, and light is irradiated from linger photoirradiation section 14.

When it is configured such that therapeutic light is emitted from finger photoirradiation section 14 regardless of whether or not the site to be treated is in contact with placement surface 22 of finger photoirradiation section 14, it is not possible to obtain an image in which the site to be treated in contact with placement surface 22 of finger photoirradiation section 14 is selectively displayed bright. This is because a portion not in contact with placement surface 22, as well as the site to be treated in contact with placement surface 22, is displayed bright. Accordingly, it is important to have a configuration such that therapeutic light is selectively irradiated onto the site to he treated in contact with finger photoirradiation section 14.

### Embodiment 2

As in Embodiment 1, Embodiment 2 has a configuration in which infrared light is used as therapeutic light, and a joint with rheumatoid arthritis developed is treated.

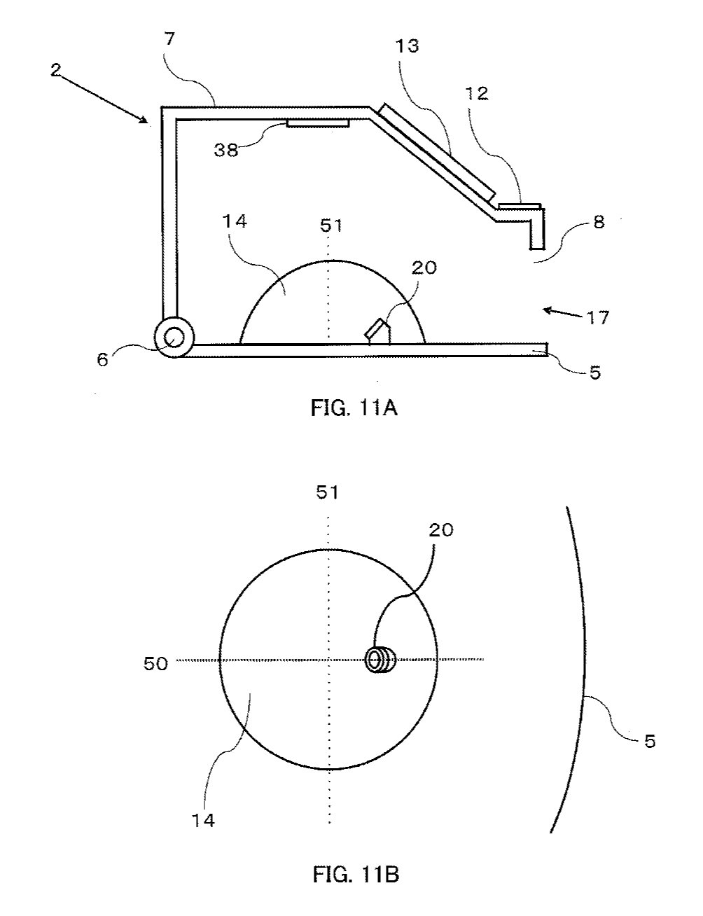

FIGS. 11A and 11B are diagrams showing the disposition of finger photographing camera 20 provided on the surface of substrate 5 of phototherapy apparatus 2 according to Embodiment 1 of the invention. FIG. 11A shows the disposition of finger photographing camera 20 when viewed from the front-back direction phototherapy apparatus 2. FIG. 11B shows the disposition of finger photographing camera 20 when viewed from the upper surface of phototherapy apparatus 2. Embodiment 2 is the same as Embodiment 1 excluding the disposition mode of finger photographing camera 20, thus description thereof will not be repeated.

It is intrinsically preferable that finger photographing camera 20 is disposed so as to photograph the entire fingers. However, in order that the entire fingers can be photographed, it is necessary to provide a sufficient distance between finger photographing camera 20 and opposite surface 23 to be photographed. As a result, finger photoirradiation section 14 increases in size, causing an increase in the size of phototherapy apparatus 2. For this reason, in order to make finger photoirradiation section 14 compact, it is necessary to contrive the disposition of finger photographing camera 20. Accordingly, in Embodiment 2, the disposition such that, instead of the entire fingers, only necessary parts are photographed by finger photographing camera 20 is shown.

When phototherapy is performed by irradiating infrared light onto the fingers of a patient with rheumatism developed, it is preferable that infrared light is intensively irradiated onto a joint from a metacarpophalangeal joint toward a fingertip. As in Embodiment 1, in phototherapy apparatus 2 of Embodiment 2, the hand is inserted from insertion slot 8, the fingers and the wrist are disposed on finger photoirradiation section 14. Accordingly, among the parts of the fingers disposed on finger photoirradiation section 14, a joint from a metacarpophalangeal joint toward a fingertip is mostly disposed in a rear portion (hinge 6 side) from the center in the front-hack direction of finger photoirradiation section 14.

Accordingly, as shown in FIGS. 11A and 11B, finger photographing camera 20 provided on the surface of substrate 5 is disposed in the front side (insertion slot 8 side) with respect to the front-back direction of finger photoirradiation section 14 so as to photograph the upper portion of opposite surface 23 on the rear side (hinge 6 side). The front side (insertion slot 8 side) refers to the side in front of center line 51 of the front-back direction. The rear side (hinge 6 side) refers to the rear portion at the back of center line 51. Finger photographing camera 20 is disposed in this way, whereby the periphery of a joint from a metacarpophalangeal joint toward a fingertip can be selectively photographed.

As shown in FIG. 11B, finger photographing camera 20 is disposed near center line 50 of the left-right direction. Accordingly, it is possible to secure a wider photographable area of finger photographing camera 20.

In Embodiment 1 and Embodiment 2, the phototherapy apparatus for treatment of a joint with rheumatism is described, and infrared light is used as therapeutic light. However, the object to be treated of the phototherapy apparatus of the invention is not limited to rheumatism, and light in other wavelength bands, such as ultraviolet light or blue light, can he of course applied as therapeutic light.

When the wavelength band of therapeutic light of light source 26 is changed, the photographing cameras (39, 40, 41, and 42) are charged to cameras which have sensitivity in the wavelength band of therapeutic light. For example, when therapeutic light is changed to ultraviolet light, a light source which output light having a principal wavelength in an ultraviolet light wavelength range is used, and an imaging camera which has sensitivity in the ultraviolet light wavelength range is used.

In Embodiment 1 and Embodiment 2, the phototherapy apparatus for treatment of a finger joint with rheumatism developed is described, and the photoirradiation section is a finger or a wrist. However, in the phototherapy apparatus of the invention, an affected part onto which light is irradiated is not limited thereto, and is appropriately selected depending on an object to be treated.

### INDUSTRIAL APPLICABILITY

The phototherapy apparatus of the invention is widely used as, for example, a phototherapy apparatus which releases pain due to chronic non-infectious inflammation, and treats a joint in which rheumatism is developed, or disease, such as psoriasis vulgaris, atopic dermatitis, vitiligo vulgaris (vitiligo), alopecia areata, prurigo, pustulosis palmoplantaris, athlete's foot, acne, or seborrhea.

### REFERENCE SIGNS LIST

1 Desk
2 Phototherapy apparatus
3 Patient
4 Chair
5 Substrate
6 Hinge
7 Cover
8 Insertion slot
9 Engagement hole
10 Hook
11 Hook button
12 Operating section
13 Display section
14 Finger photoirradiation section
15 Wrist photoirradiation section
16 Power cord
17 Hand
18 Finger
19 Wrist
20 Finger photographing camera
21 Wrist photographing camera.
22 Placement surface
23 Opposite surface
24 Light guide
2.5 Entrance
26 Light source
27 Hollow portion
28 Packing
29 Control section
30 Right finger photoirradiation section
31 Left finger photoirradiation section
32 Right wrist photoirradiation section
33 Left wrist photoirradiation section
34 Operating section
35 Buzzer
36 Opening/closing detection section
37 Apparatus internal illumination
38 Power supply section
39 Right finger photographing camera
40 Left finger photographing camera
41 Right wrist photographing camera
42 Left wrist photographing camera
43 AID converter
44 D/A converter
45 Power switch
46 Start switch
47 Treatment time input switch
48 Emergency stop switch
50 Center line of front-back direction
51 Center line of left-right direction

1. A phototherapy apparatus comprising:
a photoirradiation section that has a first surface on which a site to be treated is disposed and a second surface opposite to the first surface, and is constituted by a light guide member;
a light source section that outputs therapeutic light and inputs therapeutic light inside the photoirradiation section; and
a photographing section that has sensitivity in a wavelength band of the therapeutic light,
wherein the photographing section photographs the second surface.

2. The phototherapy apparatus according to claim 1,
wherein an affected part for treatment of the site to be treated is brought into contact with the first surface.

3. The phototherapy apparatus according to claim 1,
wherein the light guide member is made of a material having light guiding property for the therapeutic light.

4. The phototherapy apparatus according to claim 1,
wherein the light guide member is constituted by a substantially transparent member.

5. The phototherapy apparatus according to claim 1,
wherein the light source section outputs light in at least one wavelength band of infrared light, ultraviolet light, and blue light as therapeutic light.

6. The phototherapy apparatus according to claim 1.
wherein the light source section includes a plurality of light sources.

7. The phototherapy apparatus according to claim 1, further comprising:
a display section,
wherein an image photographed by the photographing section is displayed on the display section.

8. The phototherapy apparatus according to claim 1,
wherein the photoirradiation section is disposed on a substrate, the substrate surface and the photoirradiation section partition spaces to form a hollow portion, and
the photographing section is provided in the hollow portion.

9. The phototherapy apparatus according to claim 8,
wherein the hollow portion is substantially in a sealed state.

10. The phototherapy apparatus according to claim 1,
wherein the photoirradiation section has a hollow hemispherical shape.
